SEQUENCE LISTING (2 sequences)

SEQ ID NO: 1 - DNA, 50 bp, synthetic construct
  source 1..50: mol_type = other DNA
tcgtcggcagcgtcagatgtgtataagagacagcctacgggnggcwgcag

SEQ ID NO: 2 - DNA, 55 bp, synthetic construct
  source 1..55: mol_type = other DNA
gtctcgtgggctcggagatgtgtataagagacaggactachvgggtatctaatcc

(19) United States
(12) Patent Application Publication
(10) Pub. No.: US 2026/0120873 A1
(43) Pub. Date: Apr. 30, 2026

(54) METHOD FOR PREDICTING OR DIAGNOSING POST-OPERATIVE DELIRIUM USING BACTERIA-DERIVED EXTRACELLULAR VESICLES

(71) Applicant: UIF (UNIVERSITY INDUSTRY FOUNDATION), YONSEI UNIVERSITY, Seoul (KR)

(72) Inventors: Bon-Nyeo KOO, Seoul (KR); Hong KOH, Seoul (KR); Jeongmin KIM, Seoul (KR); Sujung PARK, Seoul (KR)

(73) Assignee: UIF (UNIVERSITY INDUSTRY FOUNDATION), YONSEI UNIVERSITY, Seoul (KR)

(21) Appl. No.: 19/372,764

(22) Filed: Oct. 29, 2025

(30) Foreign Application Priority Data
Oct. 29, 2024 (KR) 10-2024-0149486
Sep. 25, 2025 (KR) 10-2025-0139424

(51) Int. Cl. G16H 50/20; C12Q 1/689; G16H 50/30; G16H 50/70

(52) U.S. Cl. CPC G16H 50/20; C12Q 1/689; G16H 50/30; G16H 50/70

(57) ABSTRACT

The present disclosure relates to a method for predicting or diagnosing the occurrence of postoperative delirium using machine learning methods. The postoperative delirium prediction or diagnosis model using a random forest according to the present disclosure is developed based on selected taxa that exhibit a strong correlation between postoperative delirium and bacteria-derived extracellular vesicles, and thus can effectively diagnose and/or predict postoperative delirium.

### CROSS-REFERENCE TO RELATED APPLICATIONS

The present application claims priority to Korean Patent Application No. 10-2024-0149486, filed on Oct. 29, 2024, and Korean Patent Application No. 10-2025-0139424, filed on Sep. 25, 2025, the entire contents of which are incorporated herein by reference for all purposes.

### REFERENCE TO SEQUENCE LISTING

The instant application contains a Sequence Listing which has been submitted electronically in XML format and is hereby incorporated by reference in its entirety. Said XML copy, created on Oct. 28, 2025 is named “SOP117203US_Sequence_Listing.xml” and is 2,851 bytes in size.

### BACKGROUND OF THE INVENTION

The present disclosure relates to a method for predicting or diagnosing the occurrence of postoperative delirium using bacterial-derived extracellular vesicles and machine learning methods.

This application was supported by the Ministry of Science and ICT of the Republic of Korea under the project number 2023R1A2C1006054 (1711185524).

Delirium (postoperative delirium, POD), characterized by unpredictable progression, is a form of acute cognitive impairment. It is characterized by confusion and fluctuations in perception, orientation, memory, cognition, and behavior. Postoperative delirium is common in elderly patients after surgery and mainly appears between 2 and 52 days after surgery. In patients aged 60 years or older, postoperative delirium occurs in 20-25%. Spinal surgery accounted for 11.5% of the overall prevalence of postoperative delirium and was associated with prolonged hospital stays, increased mortality within 30 days after surgery, higher economic costs, and a higher risk of requiring a nursing facility upon discharge. As the frequency of surgery in elderly patients increases, interest in postoperative delirium is also increasing. Consequently, prediction models for POD before surgery have attracted attention. However, preoperative risk factors have been inconsistently reported in terms of sex, proinflammatory indicators, preoperative cumulative indicators, and chronic treatment. These inconsistencies in the literature have increased the likelihood of heterogeneous study cohorts, making it difficult to develop prediction models.

Delirium may be mistaken for depression due to an inactive or slow mental state. Its presentation may vary significantly among individuals from hyperactivity to hypoactivity. The heterogeneous phenotypes of delirium, along with unclear pathophysiological mechanisms, make its diagnosis, treatment, and research difficult. However, it can be prevented in approximately one-third of at-risk patients by screening individuals with risk factors and providing preoperative education. Therefore, investigating preoperative contributing factors for the postoperative delirium state is crucial for predicting clinical outcomes, and improves patient care management through intervention.

The gastrointestinal tract is generally a complex habitat inhabited by numerous microorganisms, including bacteria, viruses, and fungi. This microbiota is continuously influenced and shaped by the host and the surrounding environment, while simultaneously affecting the host's function, health, and susceptibility to disease. In animal studies, the gut microbiota has been gradually recognized as a significant contributor to postoperative mental confusion. Regular preoperative bowel preparation not only altered the gut microbiota composition in gastric cancer patients but also increased the incidence of postoperative delirium. Recent studies have shown that postoperative changes in the gut microbiota may play an important role in the development of postoperative delirium.

The gut-brain axis, which represents communication between the gut microbiota and the brain, has recently been validated through an increasing number of studies. For example, there are increasing findings that gut microbial dysbiosis can directly affect cognitive dysfunctions such as Alzheimer's disease (AD), which is caused by an increase in neurotoxic and neuroinflammatory molecules and a decrease in tryptophan- and norepinephrine-producing bacteria. In particular, patients with cognitive impairment had a decreased abundance of the anti-inflammatory genus Faecalibacterium. It has been suggested that treatments that alter the gut microbiota may help modify the neuropathology associated with AD and its progression. In contrast, the control cohort exhibited an abundance of Streptococcus equinus and Blautia hominis. These findings highlight the central role of the gut microbiota in the manifestations of postoperative delirium.

Extracellular vesicles (EVs), which are surrounded by a phospholipid bilayer membrane, are particles ranging from 20 to 400 nm in size and can be detected in all body fluids, including plasma, saliva, cerebrospinal fluid, feces, and urine. EVs are expelled from cells after their outer membrane forms a vesicle, and contain cellular proteins, lipids, bacterial DNA, and RNA. They play a crucial role in cell-to-cell communication and in promoting pathogenesis. They can enter the bloodstream and be associated with numerous host organs to modulate the immune system.

Bacteria-derived extracellular vesicles (BEVs) have been identified as potent carriers capable of crossing the blood-brain barrier and delivering signaling molecules to the central nervous system (CNS). BEVs play a role in modulating inflammation in the nervous system and also help manage tissue damage and healing. Consequently, they influence the onset, progression, and potential recovery of various diseases affecting the CNS. These diseases include autoimmune diseases, neurodegenerative diseases, stroke, traumatic brain injury, and CNS infectious diseases. Recent studies have focused on utilizing the microbiota data of serum BEVs along with clinical or pathological information to develop diagnostic tools for other diseases.

Understanding the importance of the gut-brain axis mechanism in delirium is important because it can facilitate the exploration of rational early treatment approaches. This mechanism includes direct and indirect pathways between the cognitive and emotional centers of the brain with peripheral gut functions. The gut microbiota has been recognized as a regulator of immune cells in the gut-brain communication system. The gut microbiota, that is, the microbial community living in our gut, plays an important role in this communication system. Dysbiosis of the microbiota is associated with various conditions such as depression or anxiety, and diseases related to neuroinflammation. In the field of psychiatry, active research is currently underway to explore the use of probiotics for treating and preventing microbial dysbiosis.

### PRIOR ART LITERATURE

### Patent Documents

(Patent Document 1) KR 10-2020-0073467 (Jun. 17, 2020)

### SUMMARY OF THE INVENTION

As a result of intensive efforts to provide a biomarker that can be used to screen patients with delirium by utilizing circulating bacteria-derived extracellular vesicles (BEVs), the present inventors have confirmed that postoperative delirium can be predicted when BEVs present in blood are applied to machine learning methods, thereby completing the present disclosure.

Therefore, an object of the present disclosure is to provide a method for diagnosing the occurrence of postoperative delirium by using a machine learning method, particularly a random forest.

The present disclosure provides a method for predicting or diagnosing postoperative delirium, the method comprising the steps of:
i) isolating an extracellular vesicle from a sample isolated from a preoperative subject;
ii) extracting a gene from the isolated extracellular vesicle;
iii) screening a microorganism using the extracted gene; and
iv) generating a prediction model for diagnosing postoperative delirium using a random forest for the screened microorganisms.

According to a preferred embodiment of the present disclosure, the step iii) of screening a microorganism comprises comparing a subject with postoperative delirium and a subject without postoperative delirium among the preoperative subjects in step i) to screen a microorganism with a difference in detection level.

According to a preferred embodiment of the present disclosure, the method further comprises a step v) of inputting, into the generated random forest model, the detection level of the screened microorganism in a sample isolated before surgery from a subject suspected of postoperative delirium.

According to a preferred embodiment of the present disclosure, the surgery is spinal surgery.

According to a preferred embodiment of the present disclosure, the subject is 70 years of age or older.

According to a preferred embodiment of the present disclosure, the sample is at least one selected from the group consisting of urine, feces, hair, sweat, saliva, body fluid, blood, cerebrospinal fluid, cells, and tissues.

According to a preferred embodiment of the present disclosure, the gene in step ii) is at least one selected from the group consisting of 16S rDNA, 16S rRNA, DNA, and mRNA.

According to a preferred embodiment of the present disclosure, the microorganism screened in step iv) is at least one selected from the group consisting of Moraxellaceae, Acinetobacter, Pseudomonas, Pseudomonadales, Alphaproteobacteria, Gammaproteobacteria, Bacilli, Burkholderiales, Herbaspirillum, Firmicutes, Oxalobacteraceae, Sphingomonadaceae, Sphingomonas, Sphingomonadales, Pseudomonadaceae, and Peptococcales.

The present disclosure also provides a composition for diagnosing postoperative delirium, comprising an agent capable of detecting at least one selected from the group consisting of Moraxellaceae, Acinetobacter, Pseudomonas, Pseudomonadales, Alphaproteobacteria, Gammaproteobacteria, Bacilli, Burkholderiales, Herbaspirillum, Firmicutes, Oxalobacteraceae, Sphingomonadaceae, Sphingomonas, Sphingomonadales, Pseudomonadaceae, and Peptococcales.

The present disclosure also provides a kit for diagnosing postoperative delirium, comprising the composition.

### Advantageous Effects

The postoperative delirium prediction or diagnosis model using a random forest according to the present disclosure is developed based on selected taxa that exhibit a strong correlation between postoperative delirium and bacteria-derived extracellular vesicles, and thus can effectively diagnose and/or predict postoperative delirium.

### BRIEF DESCRIPTION OF THE DRAWINGS

FIG. 1 illustrates a flow chart of the present disclosure. A total of 128 patients enrolled for spinal surgery from 2019 to 2023 were divided into two cohorts: a discovery cohort and a validation cohort. Samples from the discovery cohort were subjected to clinical testing, 16S rRNA sequencing analysis to identify and quantify bacterial taxa, and inference of functional pathways. The analyzed data were statistically screened and used to build a prediction model using a random forest classifier. The validation cohort was an independent data set that was not involved in building the prediction model to ensure the accuracy of prediction.

FIG. 2A illustrates the diversity of circulating BEVs between POD statuses. (A) The α-diversity was measured by the number of observed amplicon sequence variants (ASVs), Chao1 index representing richness, and inverse Simpson-Shannon H index representing evenness. Statistical significance between non-delirium (blue, n=45) and delirium (red, n=43) was tested by Welch's t-test. Data were expressed with individual raw data, median±interquartile range, and level of significance on top of group with higher median: *, p<0.05. (B) The β-diversity was understood by non-metric dimensional scaling (NMDS) ordinations based on Bray-Curtis dissimilarity using all ASVs. Statistical significance between non-delirium (blue, n=45) and delirium (red, n=43) was estimated by analysis of similarity (ANOSIM), resulting in p<0.01.

FIG. 2B illustrates the bacterial taxa of circulating BEVs between POD statuses. Significant bacterial taxa were organized on a cladogram based on mean fold change (FC, classified as follows: ≥4, 2-4 (including 2, excluding 4), <2) to associate clinical outcomes (i.e., blue for non-delirium and red for delirium) with bacterial lineages.

FIG. 3 illustrates the results of differential analysis for bacterial taxa isolated from BEV. Bacterial taxa recovered from circulating BEVs between patients with delirium (blue, n=45) and patients without delirium (red, n=43) were significantly different at the (A) phylum, (B) class, (C) order, (D) family, and (E) genus levels. Statistical significance between groups was tested using Welch's t-test, and the data were expressed with individual raw data, median±interquartile range, and level of significance on top of group with higher median: *, p<0.05; **, p<0.01; ***, p<0.001.

FIG. 4 shows the diversity and bacterial taxa of the gut microbiota between POD statuses. (A) α-diversity was measured by the number of observed amplicon sequence variants (ASVs), the Chao1 index for richness, and Shannon H and inverse Simpson for evenness. Statistical significance between non-delirium (blue, n=45) and delirium (red, n=43) was tested by Welch's t-test. Data were expressed with individual raw data and median±interquartile range. (B) Non-metric dimensional scaling (NMDS) order based on Bray-Curtis dissimilarity using all ASVs was used to understand β-diversity. Statistical significance between non-delirium (blue, n=45) and delirium (red, n=43) was estimated by analysis of similarity (ANOSIM). (C) Bacterial taxa extracted from stool were different significantly between non-delirium (blue, n=45) and delirium (red, n=43), which was shown at the order and lineage levels. Statistical significance between groups was tested using Welch's t-test, and the data were expressed with individual raw data and median±interquartile range. (D) Significant bacterial taxa were organized on a cladogram based on mean fold change (FC, classified as follows: ≥4, 2-4 (including 2, excluding 4), <2) to associate clinical outcomes (i.e., blue for non-delirium, red for delirium) with bacterial lineages.

FIG. 5 shows the results of the integration of significant BEV and gut microbiota. To integrate significant BEV and gut microbiota, the strength and direction of association between significant factors were measured using Pearson correlation coefficient and visualized using a heat map coded with gradient color. Blue and red indicate significant direct and inverse relationships, and blanks were found to be insignificant.

FIG. 6 shows the random forest classifier optimization and variable importance. (A) The error rates were visualized according to the number of trees and variables. A random forest classifier with 9 variables and 100 trees (black solid line) was selected for prediction model at an error rate of 21.59% (red dotted line). (B) Validity and predictive values were calculated based on the contingency table of the random forest classifier with 9 variables and 100 trees. (C) The variable importance was sorted in descending order of mean decrease in accuracy from the optimized random forest prediction model. Mean decrease accuracy is a measure of the performance of the model without each bacterial taxon, removal of high ranked taxon causes the model to lose accuracy. The two most significant variables are highlighted in red.

FIG. 7 shows the validity and predictive value of random forest models using clinical tests or gut microbiota. (A) When the prediction model was optimized using meaningful clinical tests, the random forest classifier with one variable and 200 trees (solid black line) showed an error rate of 39.77% (red dotted line). The contingency table of the random forest classifier with one variable and 200 trees (black line) was also presented along with the validity and predictive value. (B) A random forest classifier with significant gut microbiota showed an error rate of 44.32% (red dotted line) when using one variable and 400 trees (black line), and the corresponding contingency table was generated (ND, non-delirium; D, delirium; PPV, positive predictive value; NPV, negative predictive value).

FIG. 8 shows (A) the clinical outcomes of a validation cohort consisting of 27 non-delirium patients and 13 delirium patients, which were predicted by the optimized random forest classifier. The validity and predictive value of the random forest prediction model in the validation set are summarized in the contingency table. (B) Partial dependence plots were constructed to understand the relationship between the relative abundance of the two most important taxa from BEVs, i.e., Moraxellaceae and Acinetobacter, and probability of each clinical outcome, non-delirium (blue) and delirium (red). Black dotted lines indicate the minimum relative abundance at which the maximum probabilities of delirium are reached for each taxon. Additionally, the red dotted line indicates a 50% probability of outcomes. (ND, non-delirium; D, delirium; PPV, positive predictive value; NPV, negative predictive value).

FIG. 9 shows the relationship between BEV and clinical outcomes. (B) Partial dependence plots were constructed to understand the relationship between the relative abundance of significant BEVs and each clinical outcome, i.e., the probability of non-delirium (blue) and delirium (red) in (A) Phylum, (B) Class, (C) Order, (D) Family, and (E) Genus. The red dotted line indicates a 50% probability of outcomes.

FIG. 10 shows ROC curve of the abundance of Moraxellaceae and Acinetobacter BEVs for POD status. (A) ROC curve for Moraxellaceae [%] and POD outcome with AUC=0.606, optimal cut-off=3.315% (Specificity: 0.867, Sensitivity: 0.349). (B) ROC curve for Acinetobacter [%] and POD outcome with AUC=0.627, optimal cut-off=2.382% (Specificity: 0.978, Sensitivity: 0.302)

FIG. 11 shows functional pathways that exhibited statistical significance with a mean fold change of 3 or more between clinical outcomes based on 16S rRNA gene sequencing of circulating BEVs, which were considered to be significant functional pathways. Eight significant functional pathways were identified, of which five were expected to be more abundant in patients without delirium, whereas three functional pathways were found to be more abundant in patients with delirium.

FIG. 12 shows the results of differential analysis of functional pathways of BEVs. The functional pathways based on 16S rRNA gene sequencing of circulating BEVs showed statistical significance with a mean fold change of 3 or more between non-delirium (blue, n=45) and delirium (red, n=43), and were considered to be significant functional pathways, which were classified into (A) amino acid metabolism, (B) sulfur metabolism, and (C) 0-antigen biosynthesis. Statistical significance between the groups was tested using Welch's t-test, and the data were expressed with individual raw data, median±interquartile range, and level of significance on top of group with higher median (*, p<0.05; **, p<0.01).

FIG. 13 illustrates a schematic diagram of the bacterial taxa identified and inferred from BEVs and the predicted cargo metabolites.

### DETAILED DESCRIPTION

Hereinafter, the present disclosure will be described in more detail.

The term “diagnosis” in the present disclosure, in a broad sense, refers to determining the actual condition of a patient's disease in all aspects. The matters to be determined include the name of the disease, etiology, type of the disease, severity, detailed condition of symptoms, and presence or absence of complications. In the present disclosure, the diagnosis preferably refers to determining postoperative delirium and the risk of its occurrence.

The term “prognosis” in the present disclosure refers to a prospective or preliminary assessment of the medical outcome of a disease, for example, predicting a poor or good outcome (e.g., the likelihood of long-term survival). A negative prognosis or poor outcome includes a prediction of recurrence, disease progression (e.g., cancer growth or metastasis, or drug resistance), or the likelihood of death, while a positive prognosis or good outcome includes a prediction of disease cure (e.g., disease-free state), remission (e.g., eradication of cancer), or stabilization. In the present disclosure, the prognosis refers to the recurrence of postoperative delirium, overall survival, or disease-free survival. Predicting prognosis (or diagnosing prognosis) can provide clues to future treatment of postoperative delirium, including whether to receive chemotherapy, particularly in patients with early postoperative delirium. Predicting prognosis also includes predicting a patient's response to postoperative delirium treatment and the course of treatment.

The “random forest” of the present disclosure is a type of bagging algorithm consisting of a combination of CART decision trees, and was proposed by Leo Breiman and Adel Cutler. The nodes of each tree are configured to allow for rapid classification of high-dimensional data by dividing it into smaller pieces of lower dimensions. Each of these trees completes the final classification through ensemble and voting. The trees generated by random vectors with the same probability distribution are each independently constructed, and if the number of constructed trees becomes infinite, misclassifications become generalized and converge. However, the random forest uses randomness and out-of-bag (random selection without replacement) techniques to achieve accuracy comparable to Adaboost, exhibits robust performance against boundaries and noise, and help converge faster than Bagging and Boosting.

The random forest algorithm autonomously creates a plurality of (for example, 50, which can be optionally adjusted by the user) training data sets and test data sets from the given data and generates a decision tree from each. As a result, 50 independent decision trees are created. After these 50 decision trees are created, when a test set is input, each test sample has 50 decisions (delirium/non-delirium) (values from each decision tree), and the 50 decision values are filtered to obtain the final result based on the majority vote. For example, in the case of person A, if 45 decision trees judged him as delirium and 5 decision trees judged him as non-delirium, then the average score (the proportion of decisions made as delirium among the total 50 decisions) is calculated as 45/50=0.9. In this case, assuming that the cutoff value for distinguishing between delirium and non-delirium is 0.5, A's average score of 0.9 is greater than 0.5, so he can be determined to have “delirium.

Preoperative risk factors have been investigated to predict delirium after spinal surgery. Female patients, surgical history, benzodiazepine use, low hemoglobin concentration, low Mini-Mental State Examination score, high ASA score, and high C-reactive protein (CRP) were strongly associated with an increased risk of developing delirium. However, in the cohort of the present disclosure, none of the risk factors distinguished POD status. This discrepancy raises the possibility of a heterogeneous patient population with the same phenotype, and existing approaches may not accurately predict POD status. The present disclosure has focused on BEV as a new prognostic indicator based on the evidence that bacterial-derived extracellular vesicles (BEVs) harboring pathogen-associated molecular patterns (PAMPs) activate innate immunity to induce inflammatory cytokines, cross the blood-brain barrier, and exhibit changes in BEV profiles along with dysbiosis of the gut microbiota in mouse models of neurodegenerative diseases. This novel approach effectively demonstrated the core of the study, and indeed, preoperative circulating BEVs clearly distinguished postoperative delirium status in patient groups with similar baseline characteristics. This suggests that BEVs have high potential for use across a wide range of patient cohorts.

Specifically, the present disclosure aimed to elucidate the impact of blood BEV and gut microbiota composition on the development of postoperative delirium, for the purpose of establishing a predictive or diagnostic model for postoperative delirium.

A total of 128 patients were included in this study, all of whom were aged 70 years or older and scheduled for spinal surgery. Stool and serum specimens were collected immediately before surgery, and delirium was assessed at least twice daily during the postoperative hospital stay. Next-generation sequencing was utilized to analyze bacterial taxa based on 16s rRNA gene sequencing using preoperative stool samples and BEV. In order to predict postoperative delirium status using preoperative specimens, comparative analysis of significant bacterial taxonomies between non-delirium and delirium patients and a random forest classifier were employed.

Baseline characteristics of the 88 patients included in the training set were similar between the two groups. In BEV analysis, delirium patients had significantly reduced BEV diversity, lower richness (measured by observed ASV and Chao1) and lower evenness (measured by Shannon H and Inverse Simpson), compared to non-delirium patients. Clinical outcomes showed significant differences in 15 bacterial taxa present in the blood. EVs from bacteria belonging to Bacilli, Alphaproteobacteria, and Sphingomonas were more abundant in the non-delirium group, whereas BEVs from Gammaproteobacteria were more detected in the delirium group. The genera Acinetobacter, Herbaspirillum, and Pseudomonas, all belonging to the Gammaproteobacteria class, were influential variables in the model. This class includes various bacteria, including the protozoan Escherichia coli and well-known pathogens such as Salmonella, Yersinia, Vibrio, Acinetobacter, and Pseudomonas. Within the Gammaproteobacteria class, the genera Pseudomonas, Herbaspirillium, and Acinetobacter were detected at higher frequencies in the delirium group than in the non-delirium group. A significant decrease in the level of the genus Sphingomonas was observed in the delirium group. A notable characteristic of members of the Sphingomonadaceae family is the absence of lipopolysaccharides in the outer membrane, which are replaced by glycosylceramides.

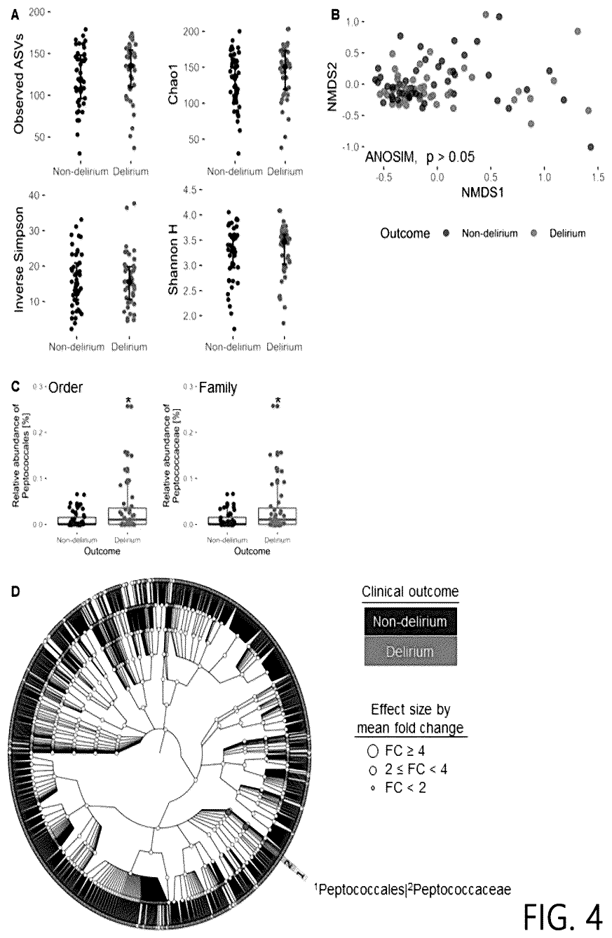

However, no significant diversity in gut microbiota or bacterial taxa were observed between the two groups. BEV analysis, rather than gut microbiota analysis, helped improve the predictive power for delirium in this study. In the gut microbiota, the Peptococcales order and Peptococccaceae family were increased in the delirium group (FIG. 4 (panel C)). The Peptococcaceae lineage includes strictly anaerobic Gram-positive cocci, and includes the genera Peptococcus, Peptostreptococcus, Ruminococcus, and Sarcina. In the present disclosure, a higher detection level of Peptococcaceae in the stool of patients before surgery was associated with a higher risk of delirium. Using functional pathway inference based on 16s rRNA gene sequence analysis, the gut environment of the non-delirium group was significantly enriched with 16 functional pathways, primarily consisting of the TCA cycle and nucleotide-related pathways (PICRUSt analysis). To understand the potential prognostic factors for predicting postoperative delirium, a prediction model was developed by applying a random forest classifier to the significant BEVs. In the model, 13 variables were identified as high-priority taxonomy, including Moraxellaceae, Acinetobacter, Pseudomonas, Alphaproteobacteria, and Gammaproteobacteria. The model achieved an accuracy of 78.41%. Furthermore, the prediction model was validated through an independent cohort, in which 80% of the patients were correctly classified.

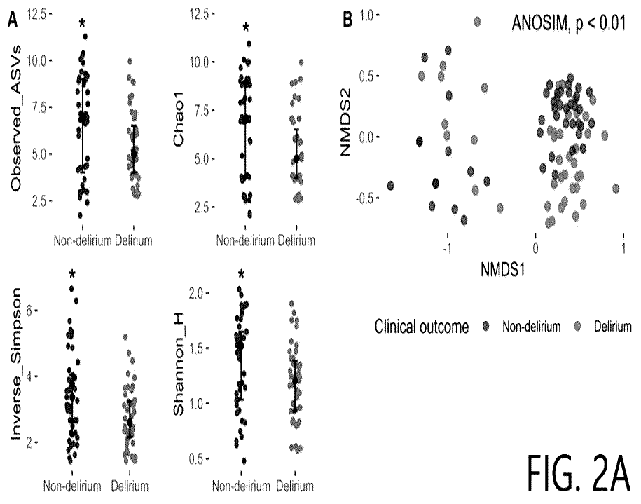

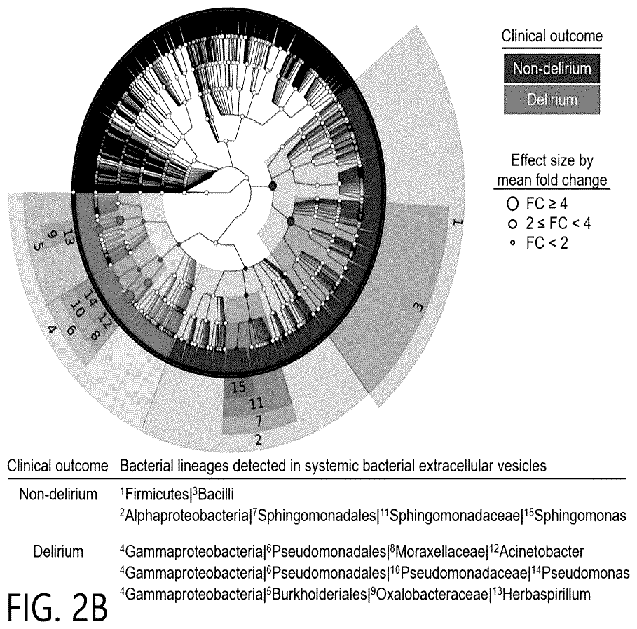

Additionally, the relative abundance of BEVs at the ASV level was shown to distinctly cluster the patient cohort into two groups, Group 1 and Group 2, clustered on the left and right, respectively (B of FIG. 2A).

Accordingly, the present disclosure provides a method for predicting or diagnosing postoperative delirium, the method comprising the steps of:
i) isolating an extracellular vesicle from a sample isolated from a preoperative subject;
ii) extracting a gene from the isolated extracellular vesicle;
iii) screening a microorganism using the extracted gene; and
iv) generating a prediction model for diagnosing postoperative delirium using a random forest for the screened microorganisms.

According to a preferred embodiment of the present disclosure, the step iii) of screening a microorganism may comprise comparing a subject with postoperative delirium and a subject without postoperative delirium among the preoperative subjects in step i) to screen a microorganism with a difference in detection level.

Specifically, the step iii) of screening a microorganism may comprise comparing a subject with postoperative delirium and a subject without postoperative delirium among the preoperative subjects in step i) to screen a microorganism with a difference in detection level of two-fold or greater in the mean fold change (FC) value.

According to a preferred embodiment of the present disclosure, the method may further comprise a step v) of inputting, into the generated random forest model, the detection level of the screened microorganism in a preoperative sample isolated from a subject suspected of postoperative delirium.

According to a preferred embodiment of the present disclosure, the surgery may be spinal surgery.

According to a preferred embodiment of the present disclosure, the subject may be 70 years of age or older.

According to a preferred embodiment of the present disclosure, the sample may be at least one selected from the group consisting of urine, feces, hair, sweat, saliva, body fluid, blood, cerebrospinal fluid, cells, and tissues. Preferably, the sample may be blood, wherein the blood may be plasma, serum, or blood cells.

According to a preferred embodiment of the present disclosure, the gene in step ii) may be at least one selected from the group consisting of 16S rDNA, 16S rRNA, DNA, and mRNA. Preferably, the gene in step ii) may be 16S rRNA.

According to a preferred embodiment of the present disclosure, the microorganism screened in step iv) may be at least one selected from the group consisting of Moraxellaceae, Acinetobacter, Pseudomonas, Pseudomonadales, Alphaproteobacteria, Gammaproteobacteria, Bacilli, Burkholderiales, Herbaspirillum, Firmicutes, Oxalobacteraceae, Sphingomonadaceae, Sphingomonas, Sphingomonadales, Pseudomonadaceae, and Peptococcales.

Preferably, the microorganism screened in step iv) may be at least one selected from the group consisting of Moraxellaceae, Acinetobacter, Pseudomonas, Pseudomonadales, Alphaproteobacteria, Gammaproteobacteria, Bacilli, Burkholderiales, Herbaspirillum, Firmicutes, Oxalobacteraceae, Sphingomonadaceae, and Sphingomonas).

The present disclosure may also provide a composition for diagnosing postoperative delirium, comprising an agent capable of detecting at least one selected from the group consisting of Moraxellaceae, Acinetobacter, Pseudomonas, Pseudomonadales, Alphaproteobacteria, Gammaproteobacteria, Bacilli, Burkholderiales, Herbaspirillum, Firmicutes, Oxalobacteraceae, Sphingomonadaceae, Sphingomonas, Sphingomonadales, Pseudomonadaceae, and Peptococcales.

Preferably, the present disclosure may provide a composition for diagnosing postoperative delirium, comprising an agent capable of detecting at least one selected from the group consisting of Moraxellaceae, Acinetobacter, Pseudomonas, Pseudomonadales, Alphaproteobacteria, Gammaproteobacteria, Bacilli, Burkholderiales, Herbaspirillum, Firmicutes, Oxalobacteraceae, Sphingomonadaceae, and Sphingomonas.

In the present disclosure, the “microorganism-detecting agent” is not limited in type of substance as long as it is an agent capable of detecting the presence of one or more selected from the group consisting of the above microorganisms for the diagnosis of postoperative delirium. For example, the microorganism-detecting agent may be an antisense oligonucleotide, primer pair, probe, peptide, polynucleotide, oligonucleotide, antibody, or aptamer that specifically binds to the 16S rRNA of the microorganism.

The detection of microorganisms using the above detecting agent may be performed by an amplification reaction using one or more oligonucleotide primers that hybridize to a nucleic acid molecule encoding a microbial-specific expression gene or a complement of the nucleic acid molecule, and the detection of nucleic acids using primers may be performed by amplifying a gene sequence using an amplification method such as PCR and then confirming whether the gene has been amplified using a method known in the art.

The “primer” refers to a short nucleic acid sequence having a short free 3′ terminal hydroxyl group, which can form base pairs with a complementary template and serves as a starting point for copying the template. In the present disclosure, the sense and antisense primers of the polynucleotide described above are used to perform PCR amplification, whereby the intermediate precursor cells can be identified based on whether a desired product is produced. The PCR conditions and the lengths of the sense and antisense primers may be modified based on those known in the art.

The “probe” refers to a nucleic acid fragment, such as RNA or DNA, ranging from a few bases to several hundred bases, that is capable of specifically binding to mRNA, wherein the probe is labeled so that the presence or absence of a specific mRNA can be identified. The probes may be prepared in the form of oligonucleotide probes, single-stranded DNA probes, double-stranded DNA probes, RNAprobes, etc. In the present disclosure, hybridization is performed using a probe complementary to the aforementioned biomarker polynucleotide, and the presence or absence of hybridization allows for the identification of intermediate precursor cells. Selection of an appropriate probe and hybridization conditions may be modified based on those known in the art.

The “aptamer” refers to a single-stranded oligonucleotide, also called a chemical antibody due to its antibody-like function, and refers to a nucleic acid molecule with binding activity to a predetermined target molecule. The aptamer may have various three-dimensional structures depending on their base sequences and exhibit high affinity for specific substances, such as antigen-antibody reactions. The aptamer can inhibit the activity of a specific target molecule by binding thereto.

The aptamer of the present disclosure may be RNA, DNA, a modified nucleic acids, or a mixture thereof, and may be a linear or cyclic form, but is not limited thereto. The aptamer having binding affinity for each of the biomarker proteins may be prepared by those skilled in the art using known methods, with reference to the respective base sequences.

The base sequence of the microorganism-detecting agent used in the present disclosure is interpreted to include sequences that exhibit substantial identity with sequences that specifically bind to microbial genes, considering variations that exhibit biologically equivalent activity. The term “substantial identity” refers to a sequence that exhibits at least 60% identity, more specifically 70% identity, even more specifically 80% identity, and most specifically 90% identity when a specific sequence and any other sequence are aligned to correspond to each other as much as possible and the aligned sequence is analyzed using an algorithm commonly used in the art.

The present disclosure may also provide a kit for diagnosing postoperative delirium, comprising the composition.

The kit of the present disclosure may be comprised of one or more other component compositions, solutions, or devices suitable for commonly used expression level analysis methods. For example, a kit for measuring protein expression levels may include a substrate, a suitable buffer, a secondary antibody labeled with a chromogenic enzyme or fluorescent substance, a chromogenic substrate, etc. for immunological detection of antibodies.

The kit of the present disclosure may include a sample extraction means for obtaining a sample from the subject to be evaluated. The sample extraction means may include a needle or a syringe, etc. The kit may include a sample collection container for receiving the extracted sample, which may be liquid, gas, or semi-solid. The kit may further include instructions for use. The sample may be any body sample in which microorganisms may be present or secreted. For example, the sample may be urine, feces, hair, sweat, saliva, body fluid, blood, cerebrospinal fluid, cells, or tissues. The detection of microorganisms in a body sample may be performed on whole or processed samples. The kit of the present disclosure may be manufactured in multiple separate packaging or compartments.

Hereinafter, the present disclosure will be described in more detail by way of examples. These examples are only for illustrating the present disclosure, and it is obvious to those skilled in the art that the scope of the present disclosure is not interpreted as limited by these examples.

### Experimental Method

### 1. Ethical Approval

This study was approved by the local Institutional Review Board (Severance Hospital 4-2019-0654, ClinicalTrials.gov identifier: NCT04120272). Written informed consent for the study was obtained. All procedures complied with the standards of the Declaration of Helsinki.

### 2. Participants

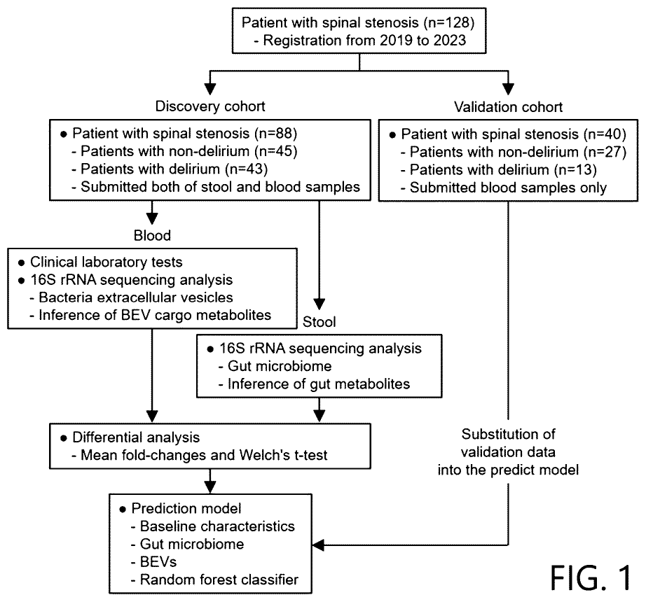

This prospective observational study was conducted at a single tertiary university hospital in Seoul, Republic of Korea. The study subjects (n=128, delirium incidence 43.8%) (Table 1) were part of the overall population (n=536, delirium incidence 17.7%) and consisted of patients aged 70 years or older who were scheduled to undergo spinal surgery between October 2019 and May 2023. A total of 128 patients were divided into a discovery cohort and a validation cohort based on the availability of samples for analysis (FIG. 1). Patients with the following conditions were excluded from the study: cognitive impairment as determined by the Mini-Mental State Examination for Dementia Screening (MMSE-DS), a diagnosis of malignancy within the past 5 years, scheduled surgery expected to take less than 2 hours, a history of neurological disease, or a diagnosis of alcoholism or drug addiction. The occurrence of POD was monitored twice daily on days 1 to 3 after surgery and once daily on days 4 to 7. When the patient showed signs of delirium according to the Confusion Assessment Method (CAM) or the Nursing Delirium Screening Scale (Nu-DESC), an experienced physician conducted further examinations to classify the patient into the delirium group.

TABLE 1

| | Non-delirium | | Delirium | | |
|---|---|---|---|---|---|
| Characteristics | (N = 72) | | (N = 56) | | p-value |
| Sex | | | | | |
| Male | 25 | (19.5%) | 17 | (13.3%) | 0.740 |
| Female | 47 | (36.7%) | 39 | (30.5%) | |
| Age [years] | 75.2 ± 4.2 | | 76.1 ± 4.4 | | 0.244 |
| Height [cm] | 158.0 ± 8.1 | | 156.6 ± 8.3 | | 0.332 |
| Weight [kg] | 60.2 ± 9.1 | | 59.2 ± 10.1 | | 0.565 |
| Body mass [kg/m²] | 24.0 ± 2.5 | | 24.1 ± 3.5 | | 0.859 |
| Surgery history | | | | | 0.788 |
| No | 10 | (7.8%) | 6 | (4.7%) | |
| Yes | 62 | (48.4%) | 50 | (39.1%) | |
| Benzodiazepine | | | | | 0.013 |
| treatment | | | | | |
| No | 69 | (53.9%) | 45 | (35.2%) | |
| Yes | 3 | (2.3%) | 11 | (8.6%) | |
| ASA-PS | | | | | 0.509 |
| I | 0 | (0.0%) | 0 | (0.0%) | |
| II | 29 | (22.6%) | 17 | (13.3%) | |
| III | 42 | (32.8%) | 38 | (29.7%) | |
| IV | 1 | (0.8%) | 1 | (0.8%) | |
| CCI | | | | | 0.662 |
| ≥4 | 5 | (3.9%) | 6 | (4.7%) | |
| <4 | 67 | (52.3%) | 50 | (39.1%) | |

| | | | |
|---|---|---|---|
| MMSE | 27.4 ± 2.0 | 27.0 ± 1.9 | 0.279 |
| MoCA | 23.7 ± 2.9 | 22.6 ± 3.4 | 0.049 |
| GDS | 4.1 ± 3.8 | 4.9 ± 4.5 | 0.293 |
| WBC [10³/μl] | 6.3 ± 1.3 | 6.9 ± 1.9 | 0.057 |
| Hemoglobin [g/dL] | 13.3 ± 1.4 | 12.9 ± 1.5 | 0.154 |
| Platelet count [10³/μl] | 237.7 ± 48.5 | 242.7 ± 65.7 | 0.631 |
| MCV[fL] | 93.0 ± 5.6 | 92.6 ± 4.6 | 0.684 |
| MCH [pg] | 30.9 ± 2.2 | 31.1 ± 1.7 | 0.623 |
| MCHC [g/dL] | 33.2 ± 1.0 | 33.6 ± 0.9 | 0.040 |
| NLR | 2.1 ± 1.4 | 2.3 ± 1.2 | 0.312 |
| LMR | 5.2 ± 2.3 | 4.8 ± 1.7 | 0.282 |
| PLR | 131.3 ± 63.2 | 135.2 ± 60.5 | 0.718 |
| ESR [ml/min/1.73 m²] | 13.8 ± 13.3 | 15.1 ± 16.1 | 0.632 |
| CRP [mg/L] | 2.4 ± 7.2 | 3.7 ± 9.0 | 0.372 |
| BUN [mg/dL] | 18.7 ± 5.2 | 22.3 ± 11.6 | 0.035 |
| Creatine [mg/dL] | 0.8 ± 0.2 | 0.9 ± 0.4 | 0.105 |
| eGFR | 77.3 ± 14.2 | 71.6 ± 18.7 | 0.060 |
| [ml/min/1.73 m²] | | | |
| Total protein [g/dL] | 7.0 ± 0.5 | 7.0 ± 0.4 | 0.901 |
| Albumin [g/dL] | 4.5 ± 0.3 | 4.4 ± 0.3 | 0.148 |

Data in [Table 1] were presented as mean±standard deviation or number of patients (percentage) (ASA-PS, the American Society of Anesthesiologists physical status classification system; CCI, Charlson Comorbidity Index; MMN/SE, Mini Mental State Examination; MoCA, Montreal Cognitive Assessment; GDS, Geriatric Depression; MCV, Mean Corpuscular Volume; MCH, Mean Corpuscular Hemoglobin; MCHC, Mean Corpuscular Hemoglobin Concentration; NLR, Neutrophil to Lymphocyte Ratio; LMR, Lymphocyte to Monocyte Ratio; PLR, Platelet to Lymphocyte Ratio; ESR, Erythrocyte Sedimentation Rate; CRP, C-Reactive Protein; BUN, Blood Urea Nitrogen; eGFR, Estimated Glomerular Filtration Rate).

### 3. Preoperative Assessment

Patients' cognitive function, depression, activities of daily living, frailty, nutritional status, and comorbidities were assessed preoperatively using validated gerontological tools. Cognitive function was evaluated using the Korean version of the MMSE-DS, and depression was evaluated using the Geriatric Depression Scale-Short Form (GDSSF-K). Functional status was assessed using the Korean version of the Activities of Daily Living Scale (K-ADL) and the Korean version of the Instrumental Activities of Daily Living Scale (K-IADL). Frailty was assessed using the FRAIL scale. Nutritional status was assessed using the Mini-Nutritional Assessment-Short Form (MNA-SF), and comorbidities were assessed using the Charlson Comorbidity Index (CCI).

### 4. Blood and Stool Sampling

5 g of stool from each patient was collected immediately before surgery using a sterilized collection device (N-Swab Transport™, NFS-2, Noble Bio, Hwacheong, Korea), immediately aliquoted into sterile cryotubes, and stored in a −80° C. freezer until DNA extraction. Blood was collected from the radial artery immediately before surgery. All blood samples were transferred to a separation tube and spun at 3,000 rpm for 15 minutes at 4° C. The clear liquid portion above the precipitate (supernatant) was collected and stored at −80° C.

### 5. Anesthesia Management

All surgeries were performed in the prone position. A Wilson frame was used with the head and neck in a neutral position. The types of surgery included laminectomy, discectomy, and spinal fusion. Anesthesia was induced with propofol (1-1.5 mg/kg), remifentanil (0.05-0.2 g/kg/min), and rocuronium (0.6 mg/kg). Anesthesia was maintained using inhalation or intravenous anesthesia. During surgery, the concentration of sevoflurane, desflurane, or propofol was adjusted to achieve a SedLine® Patient State Index (PSI) of 25-50, which is recommended by the manufacturer for anesthesia induction in general surgical patients to ensure safety and efficacy. Vasoactive drugs such as norepinephrine and ephedrine were used to maintain mean blood pressure within 80-120% of baseline during surgery. The lungs were ventilated with a 50% oxygen/air mixture.

6. Genomic DNA Extraction from Stool Samples

Total genomic DNA was extracted from 0.2 g of stool samples using the Maxwell CSC PureFood GMO and Authentication Kit (Promega, USA) according to the manufacturer's instructions. DNA concentration was measured using a UV-vis spectrophotometer (NanoDrop 2000c, USA), and DNA quantification was performed using the QuantiFluor ONE dsDNA System (Promega, USA). All extracted DNA was stored at −20° C. until used for further experiments.

### 7. Analysis of the Gut Environment

The composition of the microbiota was analyzed by 16S rRNA amplicon sequencing using Illumina MiSeq (Illumina, Inc., USA). For sequence analysis, the V3-V4 regions of the bacterial 16S rRNA gene were amplified using primer sets F319 and R806. All procedures were conducted in accordance with the manufacturer's protocols provided by Illumina.

Analysis of the gut microbiota was performed using the QIIME 2 2022.02 pipeline. Paired-end sequence data were demultiplexed using MiSeq Reporter and merged using the q2-vsearch plugin. The merged sequences were quality-filtered using the q2-quality-filter plugin and then denoised with Deblur (via q2-deblur). Classification was assigned to ASVs using the q2feature-classifier classify-sklearn naive Bayes classifier for SILVA DB v138. Functional pathways were analyzed by phylogenetic investigations using PICRUSt (Reconstruction of Unobserved States) 2 v2.3.0 beta based on 16S rRNA gene sequences. This allowed the present inventors to predict the metagenomic content up to MetaCyc to infer a functional pathway.

### 8. Analysis of Bacterial Extracellular Vesicles

Nanovesicles were isolated from the samples via differential centrifugation, and genomic DNA was extracted. Then, 16S rRNA sequencing was performed using an Illumina MiSeq (Illumina, USA). Through this process, the gut microbiota was classified, and correlations between clinical characteristics and the abundance of specific microorganism-derived rRNA were derived (MD Healthcare, Seoul, Korea).

Bacterial EVs were boiled at 100° C. for 40 minutes using a heat block, and then the remaining particles and waste were removed by centrifugation at 18,312 g for 30 minutes at 4° C. DNA was extracted from the supernatant using the DNeasy PowerSoil Pro kit (QIAGEN, Germany). The DNA of bacterial EVs in each sample was quantified using QIAxpert (QIAGEN, Germany). The V3-V4 region of the 16S rDNA gene was amplified using primers. 16S_V3_F (5′-TCG TCG GCA GCG TCA GAT GTG TAT AAG AGA CAG CCT ACG GGN GGC WGC AG-3′, SEQ ID NO: 1) and 16S_V4_R (5′-GTC TCG TGG GCT CGG AGA TGT GTA TAA GAG ACA GGA CTA CHV GGG TAT CTA ATC C-3′, SEQ ID NO: 2). Library preparation was performed using PCR products, and each amplicon was sequenced by MiSeq.

The Illumina output, which included both the nucleotide sequence of the reads and the quality score (Q-score) associated with each nucleotide in each read, was imported to QIIME2 (https://qiime2.org/2021.4/). After removal of the V3-V4 primers, forward and reverse reads were truncated at 200 and 260 bases, respectively, based on Q-scores. DADA2 defaulted the action regarding the chimera to “consensus” and pooling to “independent.” The DADA2 algorithm is used to model and correct errors in Illumina-sequenced amplicons and to identify ASVs. In this study, a naïve Bayesian classifier was pretrained on the SILVA 138 database and then used for taxonomic annotation of samples. Samples with fewer than 1,000 reads were not considered for downstream analysis.

### 9. Functional Pathway Inference

To infer metabolites transferred from the gut environment or BEV cargo, functional pathways were analyzed using Phylogenetic Investigation of Communities by Reconstruction of Unobserved States (PICRUSt) 2 v2.3.0 beta based on 16S rRNA gene sequences. This allowed for the prediction of metagenome content up to MetaCyc, enabling the inference of functional pathways. Functional pathways with a mean fold change (ratio of relative abundance of functional pathways in non-delirium and delirium patients) of ≥3 and a p<0.05 between clinical outcomes were considered significant functional pathways.

### 10. Statistical Analysis

In the data preprocessing step, missing values were imputed with predicted values using the proximity matrix of a random forest (rflmpute( ) function in the randomForest R package). For alpha diversity analysis, 16s rRNA sequence reads were rarefied to standardize the sequencing depth of each sample to the minimum read count (Rarefy(depth=min( )) function in the GUniFrac R package). Subsequently, alpha diversity by group was measured by observed ASVs (specnumber( ) function in the vegan R package) and Chao1 (estimate(permutations=100) function) for species richness, and Shannon H (diversity(index=“simpson”) in the vegan R package) and Inverse Simpson (diversity(index=“invsimpson”) in the vegan R package) for evenness.

In beta diversity analysis, sequence reads were normalized to relative abundance (the ratio of each taxon to the sum of all observed taxa in the feature table) to analyze intergroup diversity. The results were visualized using non-metric multidimensional scaling (NMDS) based on Bray-Curtis dissimilarity (via metaMDS(distance=“bray”) function), and significance was tested using analysis of similarity (ANOSIM) (anosim(distance=“bray”, permutations=9999) function in the vegan R package). Bacterial taxa that were significant for each group were plotted as a circular cladogram using GraPhlAn to understand the signature bacterial lineages associated with each clinical outcome.

Statistical comparison of variables between the two groups was performed using Welch's t-test (t.test(var.equal=FALSE) in the stats R package) for continuous variables and chi-square test (chisq.test( ) in the stats R package) for categorical variables (FIG. 1). Variables that showed significant differences between the two groups and no redundancy were used to build decision trees using the random forest machine learning algorithm (randomForest(importance=TRUE, proximity=TRUE) in the randomForest R package). Optimization of the random forest model was based on the lowest out-of-bag (OOB) score to determine the number of trees and variables (FIG. 1). To evaluate the model's accuracy in predicting clinical outcomes, the error rate, accuracy, sensitivity, specificity, positive and negative predictive values were calculated (confusionMatrixo function in the caret R package). Additionally, a partial dependence plot was created to understand how changes in specific feature values affect the random forest model predictions (partial_dependence( ) function in the edarF R package).

To integrate significant factors from blood and stool samples, the strength and direction of associations between factors were measured using Pearson's correlation coefficient and p-value, and visualized using correlograms (using cor_mat(method=“pearson”) and cor_plot in the rstatix R package).

All statistical analyses were performed at a significance level of 5% using R version 4.4.2 (R Core Team, R Foundation for Statistical Computing, Vienna, Austria).

### Example 1

### Confirmation of Baseline Characteristics of Non-Delirium and Delirium Patients

The discovery cohort included 88 patients who underwent spinal surgery. 45 patients did not develop delirium, but 43 patients (48.9%) developed delirium at a mean of 67.5 hours (median=48 hours, minimum=24 hours, maximum=216 hours) after spinal surgery. Baseline characteristics, which are commonly considered preoperative risk factors, were strongly associated with POD. Except for white blood cell (WBC) count and mean corpuscular hemoglobin concentration (MCHC), no significant differences were observed between patients with and without delirium. Preoperative WBC and triglyceride levels were significantly higher in patients with delirium compared to patients without delirium. However, considering the normal ranges for WBC (4.5-11.0×10³/μL) and triglyceride (32-36 g/dL), it is likely that these statistical differences do not explain actual clinical differences. Overall, the two groups were similar in demographic characteristics, anthropometric measurements, cognitive function scores, and clinical test indicators because the study cohort was composed by matching the non-delirium group to the delirium group based on major prognostic factors known to influence the development of delirium. For these reasons, it was not possible to find factors showing significant differences using only the existing approach, and therefore, in this cohort, discovering new factors significantly associated with postoperative delirium will be a key strategy for subsequent analyses.

### Example 2

### Confirmation of Association Between Preoperative Blood-Derived Bacterial Taxa and Postoperative Mental Confusion

Recently, BEVs have been considered to transmit messages from the gut environment to extraintestinal organs, including the brain. To understand BEVs as a potential prognostic factor for postoperative delirium, the present inventors sequenced 16S rRNA genes obtained from blood samples associated with clinical outcomes.

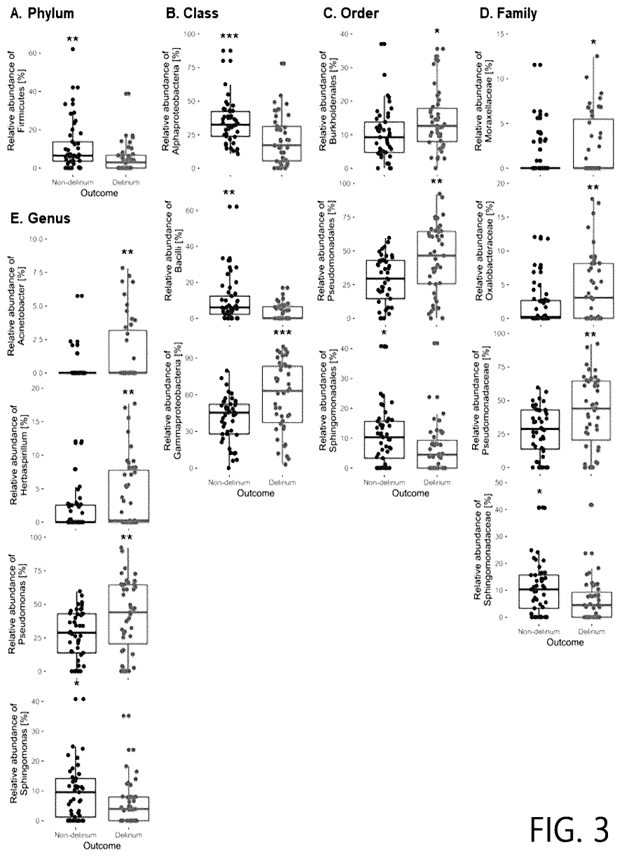

Regarding the diversity of BEVs within groups (A of FIG. 2A), patients with delirium had lower richness (measured by observed amplicon sequence variation (ASV) and Chao1) and evenness (measured by Shannon H and Inverse Simpson) compared to patients without delirium. This indicates lower numbers and diversity of BEVs in the preoperative blood of patients with delirium. As a result of using a non-metric multidimensional scaling method that uses all ASVs to determine the diversity of BEVs between groups, the composition of systemic BEVs was different significantly between patients with and without delirium (B of FIG. 2A). To correlate BEVs with POD status, significantly different bacterial taxa extracted from blood samples (FIG. 3) were visualized in a cladogram (FIG. 2B). At the class level, BEVs derived from Bacilli and Alphaproteobacteria were detected in greater abundance in the group without delirium, whereas BEVs derived from Gammaproteobacteria were more detected in the group with delirium (FIG. 2B). In particular, the genus Acinetobacter of the Gammaproteobacteria lineage most significantly discriminated clinical prognosis. The relative abundance in delirium was at least four times higher than in the group without delirium (FIG. 2B). These data showed that the pattern of BEV in preoperative blood was significantly associated with POD status.

### Example 3

### Confirmation of Relationship Between Systemic BEV and Gut Microbiota

To connect the results of BEV with the gut microbiota, the inventors analyzed the gut environment, including gut microbiota and functional pathways, under the hypothesis that gut microbial taxa are similar to the bacterial taxa rescued from systemic BEVs.

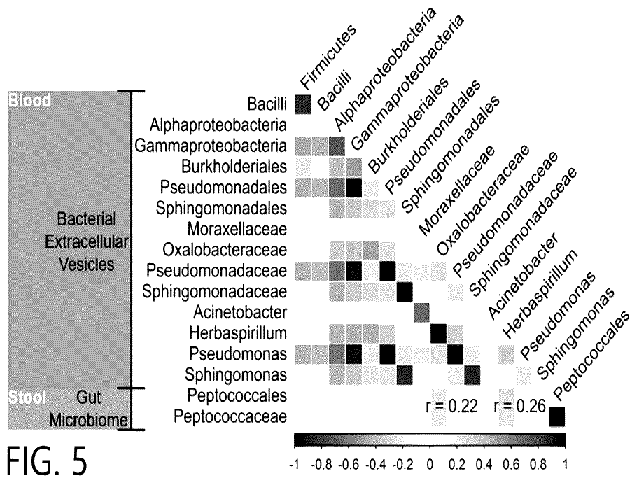

As a result, for microbial diversity analysis, intra-group diversity and inter-group diversity (FIG. 4 panel A and FIG. 4 panel B) did not reach statistical significance. Furthermore, gut bacterial taxa had little association with clinical outcomes. Only two taxa, the Peptococcales order and the Peptococcaceae family, were more detected in delirium patients than in non-delirium patients (FIG. 4 panel C and FIG. 4 panel D). The strength and direction of association between significant BEV and gut microbiota were measured using Pearson's correlation, and no strong correlation was found. If a correlation were present, the coefficients were only 0.22 and 0.26 (FIG. 5). Therefore, the profile of systemic BEV of the subject population of the present disclosure was not associated with the profile of the gut microbiota.

### Example 4

Random Forest Model with Significant BEV for Predicting Postoperative Delirium

Applying machine learning algorithms to predict clinical outcomes before intervention has made significant contributions for patients. A random forest classifier was used together with significant factors to build a prediction model for postoperative delirium status. EVs from Sphingomonadales, Pseudomonadaceae, and Peptococcales were not considered in the prediction model due to redundancy.

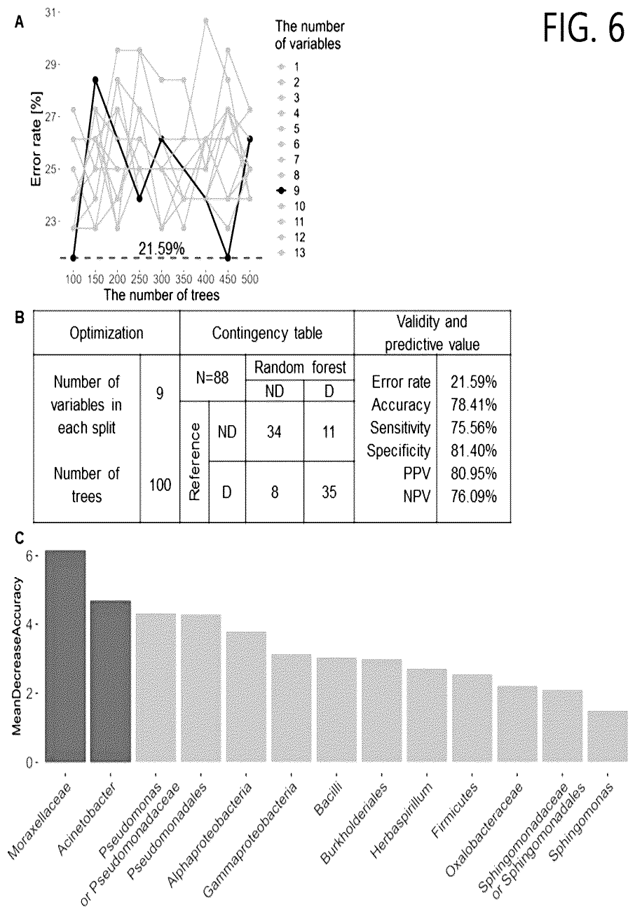

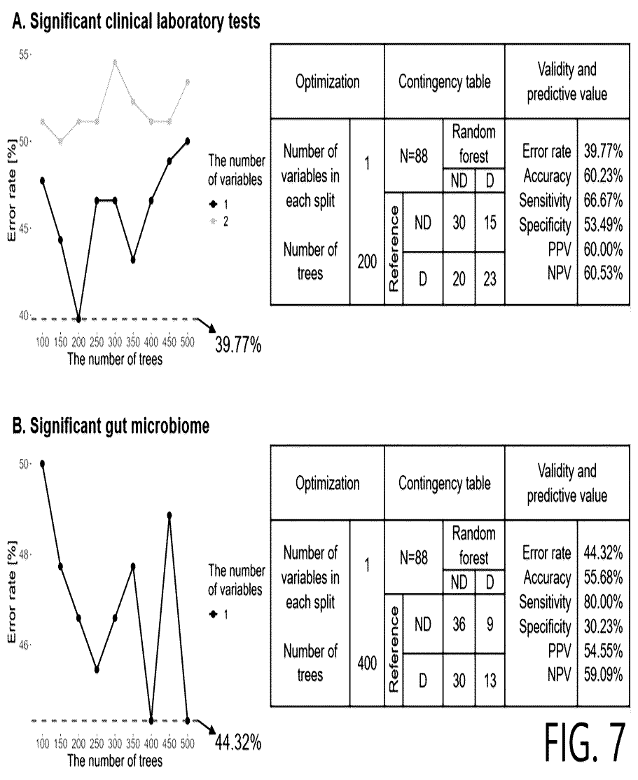

Compared to clinical laboratory testing (FIG. 7 panel A) or gut microbiome (FIG. 7 panel B) factors, the random forest classifier using BEV showed the lowest prediction error rate of 21.59%0 as measured by the out-of-bag (GOB) error for 100 trees. To understand which feature is most important for the prediction model of nine variables (FIG. 6 panel A; Moraxellaceae, Acinetobacter, Pseudomonas, Pseudomonadales, Alphaproteobacteria, Gammaproteobacteria, Bacilli, Burkholderiales, and Herbaspirillum), the average decrease in accuracy was reported across all trees for 13 significant factors, with Moraxellaceae and Acinetobactor being the top two significant features across 100 decision trees (FIG. 6 panel C).

TABLE 2

| | Non-delirium | | Delirium | | |
|---|---|---|---|---|---|
| Characteristics | (N = 27) | | (N = 13) | | p-value |
| Sex | | | | | |
| Male | 13 | (32.5%) | 3 | (7.5%) | 0.241 |
| Female | 14 | (35.0%) | 10 | (25.0%) | |

| Age [years] | 75.2 ± 4.9 | | 78 ± 5.8 | | 0.145 |
|---|---|---|---|---|---|
| Height [cm] | 158.7 ± 8.6 | | 154.2 ± 10.0 | | 0.175 |
| Weight [kg] | 59.4 ± 8.7 | | 55.2 ± 10.2 | | 0.212 |
| Body mass [kg/m²] | 23.5 ± 2.1 | | 23.1 ± 3.1 | | 0.691 |
| Surgery history | | | | | 0.671 |
| No | 5 | (12.5%) | 1 | (2.5%) | |
| Yes | 22 | (55.0%) | 12 | (30.0%) | |
| Benzodiazepine | | | | | 0.048 |
| treatment | | | | | |
| No | 25 | (62.5%) | 8 | (20.0%) | |
| Yes | 2 | (5.0%) | 5 | (12.5%) | |
| ASA-PS | | | | | 0.386 |
| I | 0 | (0.0%) | 0 | (0.0%) | |
| II | 11 | (27.5%) | 3 | (7.5%) | |
| III | 15 | (37.5%) | 10 | (25.0%) | |
| IV | 1 | (2.5%) | 0 | (0.0%) | |
| CCI | | | | | 0.448 |
| ≥4 | 4 | (10.0%) | 4 | (10.0%) | |
| <4 | 23 | (57.5%) | 9 | (22.5%) | |

| | | | |
|---|---|---|---|
| MMSE | 26.9 ± 2.2 | 25.4 ± 2.1 | 0.046 |
| MoCA | 23.2 ± 3.4 | 19.7 ± 3.5 | 0.007 |
| GDS | 4.9 ± 3.8 | 7.3 ± 4.5 | 0.116 |
| WBC [10³/μl] | 6.6 ± 1.4 | 7.2 ± 2.4 | 0.409 |
| Hemoglobin [g/dL] | 13.6 ± 1.3 | 12.0 ± 0.8 | <0.0001 |
| Platelet count [10³/μl] | 231.1 ± 47.5 | 285.1 ± 93.8 | 0.069 |
| MCV[fL] | 94.6 ± 3.9 | 92.0 ± 6.0 | 0.177 |
| MCH [pg] | 31.6 ± 1.3 | 30.7 ± 2.1 | 0.181 |
| MCHC [g/dL] | 33.4 ± 0.7 | 33.3 ± 0.6 | 0.731 |
| NLR | 2.1 ± 1.1 | 2.7 ± 1.4 | 0.200 |
| LMR | 4.9 ± 2.0 | 3.6 ± 0.9 | 0.013 |
| PLR | 122.8 ± 46.2 | 169.7 ± 80.0 | 0.067 |
| ESR [ml/min/1.73 m²] | 11.7 ± 9.7 | 18.7 ± 16.6 | 0.176 |
| CRP [mg/L] | 2.1 ± 5.0 | 6.2 ± 15.9 | 0.387 |
| BUN [mg/dL] | 17.5 ± 4.4 | 21.9 ± 8.1 | 0.087 |
| Creatine [mg/dL] | 0.8 ± 0.2 | 0.8 ± 0.2 | 0.669 |
| eGFR [ml/min/1.73 m²] | 82.0 ± 11.0 | 74.5 ± 18.2 | 0.190 |
| Total protein [g/dL] | 7.0 ± 0.3 | 6.8 ± 0.3 | 0.032 |
| Albumin [g/dL] | 4.4 ± 0.2 | 4.3 ± 0.3 | 0.464 |

Data in [Table 2] were presented as mean±standard deviation or number of patients (percentage) (ASA-PS, the American Society of Anesthesiologists physical status classification system; CCI, Charlson Comorbidity Index; MMSE, Mini Mental State Examination; MoCA, Montreal Cognitive Assessment; GDS, Geriatric Depression; MCV, Mean Corpuscular Volume; MCH, Mean Corpuscular Hemoglobin; MCHC, Mean Corpuscular Hemoglobin Concentration; NLR, Neutrophil to Lymphocyte Ratio; LMR, Lymphocyte to Monocyte Ratio; PLR, Platelet to Lymphocyte Ratio; ESR, Erythrocyte Sedimentation Rate; CRP, C-Reactive Protein; BUN, Blood Urea Nitrogen; eGFR, Estimated Glomerular Filtration Rate).

TABLE 3

| | | Random forest | | Reference = |
|---|---|---|---|---|
| Patient ID | Reference | prediction | Vote [%] | Prediction |
| 1 | Delirium | Delirium | 79 | Y |
| 2 | Non-delirium | Non-delirium | 90 | Y |
| 3 | Delirium | Delirium | 69 | Y |
| 4 | Delirium | Non-delirium | 64 | N |
| 5 | Delirium | Delirium | 58 | Y |
| 6 | Delirium | Delirium | 67 | Y |
| 7 | Delirium | Non-delirium | 60 | N |
| 8 | Delirium | Non-delirium | 80 | N |
| 9 | Delirium | Delirium | 96 | Y |
| 10 | Delirium | Delirium | 58 | Y |
| 11 | Delirium | Delirium | 68 | Y |
| 12 | Delirium | Delirium | 50 | Y |
| 13 | Delirium | Delirium | 97 | Y |
| 14 | Non-delirium | Non-delirium | 76 | Y |
| 15 | Non-delirium | Delirium | 59 | N |
| 16 | Non-delirium | Non-delirium | 72 | Y |
| 17 | Non-delirium | Delirium | 77 | N |
| 18 | Non-delirium | Delirium | 68 | N |
| 19 | Non-delirium | Delirium | 94 | N |
| 20 | Non-delirium | Non-delirium | 80 | Y |
| 21 | Non-delirium | Delirium | 84 | N |
| 22 | Non-delirium | Non-delirium | 63 | Y |
| 23 | Non-delirium | Non-delirium | 74 | Y |
| 24 | Non-delirium | Non-delirium | 75 | Y |
| 25 | Non-delirium | Non-delirium | 73 | Y |
| 26 | Non-delirium | Non-delirium | 54 | Y |
| 27 | Non-delirium | Non-delirium | 72 | Y |
| 28 | Non-delirium | Non-delirium | 73 | Y |
| 29 | Non-delirium | Non-delirium | 74 | Y |
| 30 | Non-delirium | Non-delirium | 84 | Y |
| 31 | Non-delirium | Non-delirium | 78 | Y |
| 32 | Non-delirium | Non-delirium | 74 | Y |
| 33 | Non-delirium | Non-delirium | 77 | Y |
| 34 | Non-delirium | Non-delirium | 51 | Y |
| 35 | Non-delirium | Non-delirium | 98 | Y |
| 36 | Non-delirium | Non-delirium | 69 | Y |
| 37 | Non-delirium | Non-delirium | 75 | Y |
| 38 | Non-delirium | Non-delirium | 52 | Y |
| 39 | Non-delirium | Non-delirium | 93 | Y |
| 40 | Delirium | Delirium | 89 | Y |

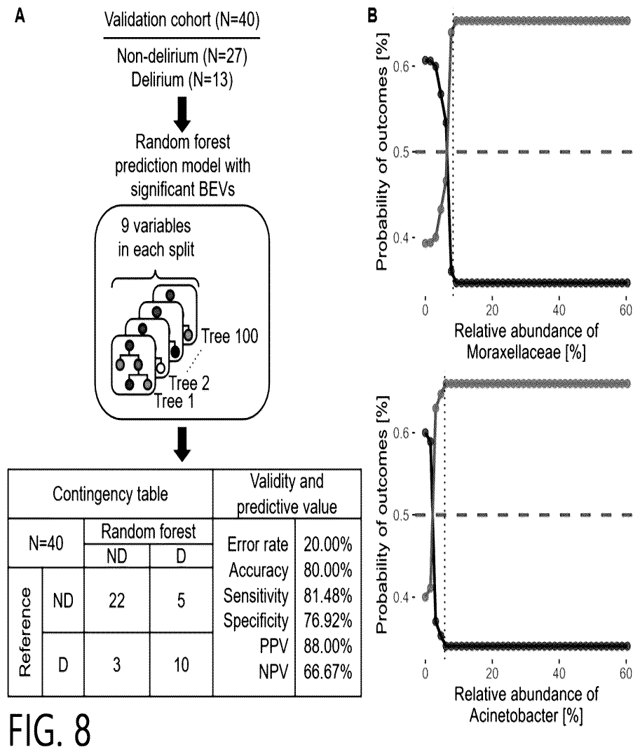

To apply the prediction model described above to a clinical setting, it was important to validate the model using an independent dataset. To this end, the prediction model was validated using an external dataset composed of 40 patients. 13 patients exhibited postoperative delirium, while 27 patients did not (Table 2). The prediction model correctly classified 32 out of the 40 patients but misclassified 8, resulting in an accuracy of 80.0000, an error rate of 20.00%, a sensitivity of 71.48%, a specificity of 66.920%, a positive predictive value (PPV) of 88.000%, and a negative predictive value (NPV) of 66.67% (FIG. 8 panel A, Table 3).

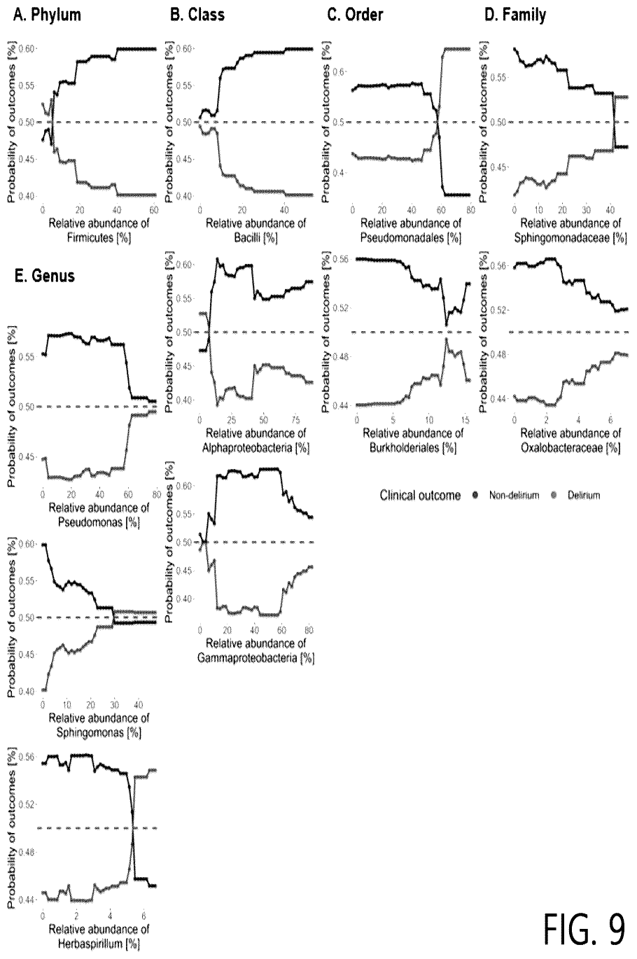

To understand how the relative abundance of BEV affected the predictions of the random forest, partial dependence plots were constructed using each significant bacterial taxon (FIG. 8 panel B, FIG. 9). Among the significant factors, two taxa from BEVs showed the highest probability of predicting POD status, despite their low relative abundance. Patients with ≥5.8% Acinetobacter EV and ≥8.3% Moraxellaceae EV in their preoperative blood samples (black dotted lines) had a higher likelihood of developing postoperative delirium, with probabilities of 66% and 65%, respectively (FIG. 8 panel B). These data suggested that preoperative circulating EVs derived from Moraxellaceae or Acinetobacter were the most important prognostic indicators of POD.

### Example 5

### Cut-Off Values of the Most Significant Prognostic BEVs for POD

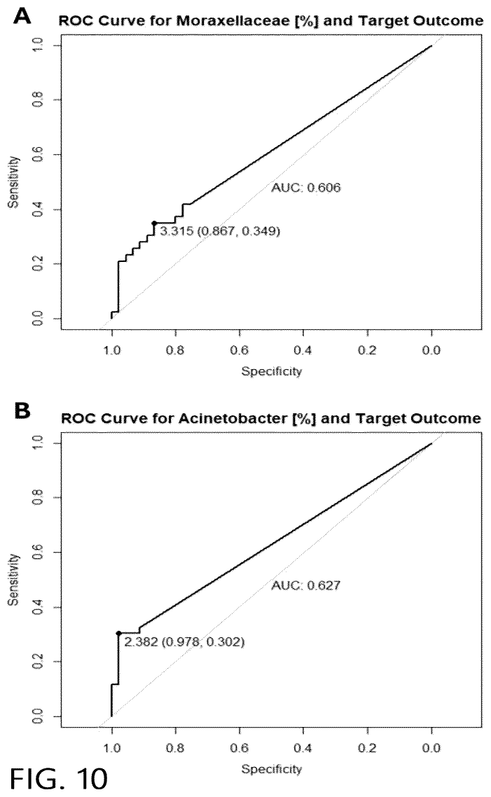

ROC curve, AUC value and optimal threshold point with corresponding sensitivity and specificity were generated with the relative abundance of Moraxellaceae (FIG. 10 panel A) and Acinetobacter (FIG. 10 panel B) BEVs in relation to POD status via pROC R package.

The optimal cut-off values for predicting POD were determined from ROC analysis; blood samples with Moraxellaceae≥3.315% (FIG. 10 panel A) or Acinetobacter≥2.382% (FIG. 10 panel B) are more likely to be associated with the POD.

### Example 6

### BEV Cargo Metabolites Regulating POD Status

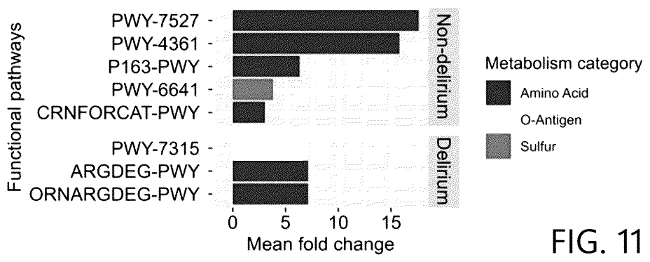

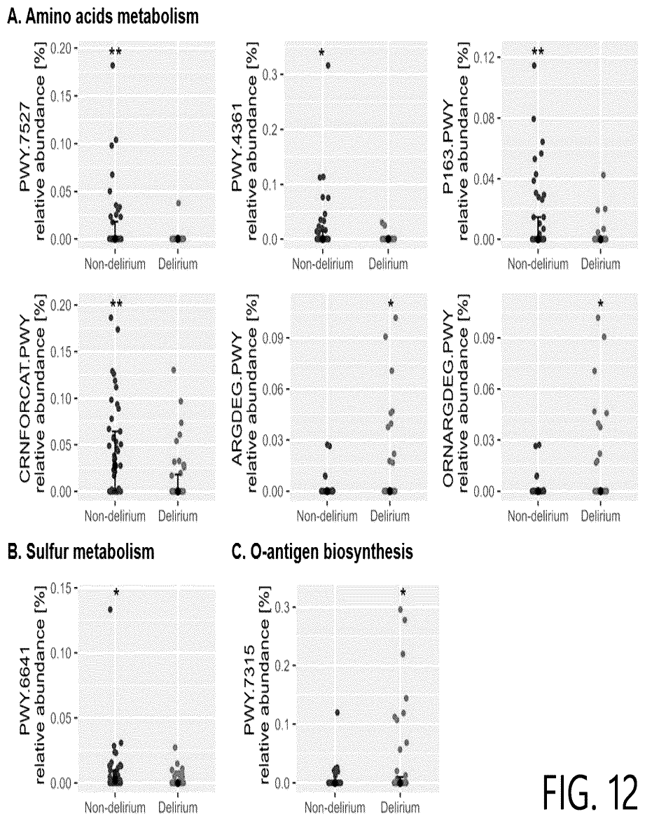

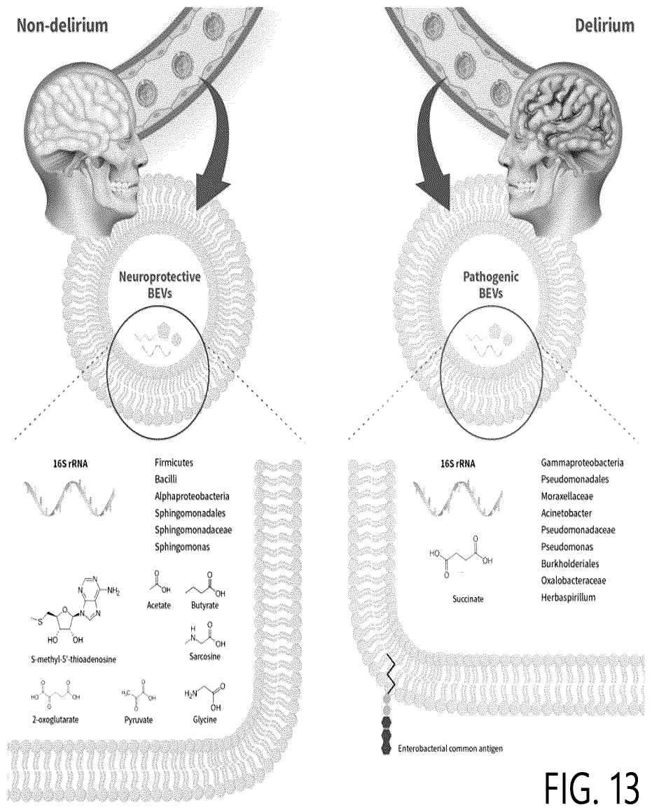

To understand the potential mechanisms by which BEV influences POD status, the inventors inferred cargo metabolites that the BEV can deliver based on the analysis of 16s rRNA gene sequencing of blood samples, and aggregated the relative abundance of functional genes into metabolic pathways. Patients with non-delirium and delirium were expected to be associated with five and three functional pathways, respectively (FIG. 11 and FIG. 12). Considering the metabolites produced from the pathways, nine metabolites were expected to regulate the clinical outcomes. S-methyl-5′-thioadenosine (MTA) from PWY-7527, 2-oxoglutarate from PWY-4361, acetate and butyrate from P163-PWY, pyruvate from PWY-6641, and sarcosine and glycine from CRNFORCAT-PWY appeared to be neuroprotective, whereas accumulation of succinate from ORNARGDEG-PWY and ARGDEG-PWY, and enterobacterial common antigen from PWY-7315 may participate in pathogenic mechanisms of the POD (FIG. 13). The profile of metabolites inferred in the present disclosure can be utilized as useful research resource to investigate defensive and offensive molecular mechanisms in each clinical outcome.

What is claimed is:

1. A method for predicting or diagnosing postoperative delirium, the method comprising the steps of:
i) isolating an extracellular vesicle from a sample isolated from a preoperative subject;
ii) extracting a gene from the isolated extracellular vesicle;
iii) screening a microorganism using the extracted gene; and
iv) generating a prediction model for diagnosing postoperative delirium using a random forest for the screened microorganisms.

2. The method of claim 1, wherein the step iii) of screening a microorganism comprises comparing a subject with postoperative delirium and a subject without postoperative delirium among the preoperative subjects in step i) to screen a microorganism with a difference in detection level.

3. The method of claim 1, further comprising a step v) of inputting, into the generated random forest model, the detection level of the screened microorganism in a sample isolated before surgery from a subject suspected of postoperative delirium.

4. The method of claim 1, wherein the surgery is spinal surgery.

5. The method of claim 1, wherein the subject is 70 years of age or older.

6. The method of claim 1, wherein the sample is at least one selected from the group consisting of urine, feces, hair, sweat, saliva, body fluid, blood, cerebrospinal fluid, cells, and tissues.

7. The method of claim 1, wherein the gene in step ii) is at least one selected from the group consisting of 16S rDNA, 16S rRNA, DNA, and mRNA.

8. The method of claim 1, wherein the microorganism screened in step iv) is at least one selected from the group consisting of Moraxellaceae, Acinetobacter, Pseudomonas, Pseudomonadales, Alphaproteobacteria, Gammaproteobacteria, Bacilli, Burkholderiales, Herbaspirillum, Firmicutes, Oxalobacteraceae, Sphingomonadaceae, Sphingomonas, Sphingomonadales, Pseudomonadaceae, and Peptococcales.

9. A composition for diagnosing postoperative delirium, comprising an agent capable of detecting at least one selected from the group consisting of Moraxellaceae, Acinetobacter, Pseudomonas, Pseudomonadales, Alphaproteobacteria, Gammaproteobacteria, Bacilli, Burkholderiales, Herbaspirillum, Firmicutes, Oxalobacteraceae, Sphingomonadaceae, Sphingomonas, Sphingomonadales, Pseudomonadaceae, and Peptococcales.

10. A kit for diagnosing postoperative delirium, comprising the composition of claim 9.
